(19) United States
(12) Patent Application Publication
(10) Pub. No.: US 2025/0152957 A1
(43) Pub. Date: May 15, 2025

(54) DRIVING METHOD OF TRANSCRANIAL MAGNETIC STIMULATION 3D COIL DEVICE

(71) Applicant: Metal Industries Research & Development Centre, Kaohsiung (TW)

(72) Inventors: Zong-Hsin Liu, Kaohsiung City (TW); Po-Chi Hu, Kaohsiung City (TW); Chih-Chung Lin, Kaohsiung City (TW); Chih-Hsuan Huang, Kaohsiung City (TW); I-Chiao Tsai, Kaohsiung City (TW); Jia Yan Lin, Kaohsiung City (TW); Yan Ting Chen, Kaohsiung City (TW); Chieh Hua Chen, Kaohsiung City (TW)

(73) Assignee: Metal Industries Research & Development Centre, Kaohsiung (TW)

(21) Appl. No.: 18/523,849

(22) Filed: Nov. 29, 2023

(30) Foreign Application Priority Data
Nov. 10, 2023 (TW) 112143422

(51) Int. Cl. A61N 2/02; A61N 2/00; H01F 7/06

(52) U.S. Cl. CPC A61N 2/02; A61N 2/006; H01F 7/064

(57) ABSTRACT

A driving method of a transcranial magnetic stimulation 3D coil device is disclosed, which includes: determining first and second signal peaks corresponding to a specified rotating magnetic field direction, in which the first and second signal peaks respectively correspond to first and second coils in multiple coils; providing a first pulse current having the first signal peak to the first coil, in which the first pulse current signal stimulates the first coil to provide a first magnetic field; providing a second pulse current signal having a second signal peak to the second coil, in which the second pulse current signal stimulates the second coil to provide a second magnetic field, in which the first magnetic field and the second magnetic field form a rotating magnetic field having the specified rotating magnetic field direction.

### CROSS-REFERENCE TO RELATED APPLICATION

This application claims the priority benefit of Taiwan application serial no. 112143422, filed on Nov. 10, 2023. The entirety of the above-mentioned patent application is hereby incorporated by reference herein and made a part of this specification.

### BACKGROUND

### Technical Field

The disclosure relates to a transcranial magnetic stimulation (TMS) technology, and particularly relates to a transcranial magnetic stimulation three-dimensional (3D) coil device and a driving method thereof.

### Description of Related Art

In the existing technology, it is a very common medical method to treat patients with depression or other brain diseases by using a transcranial magnetic stimulation (TMS) technology.

For example, US patent publication no. US20210228898A1 discloses an 8-shaped TMS device that includes an electromagnet, a driving circuit electrically coupled to the electromagnet, and a controller configured to control the driving circuit to provide current to the electromagnet to produce a pulse magnetic field.

However, the above 8-shaped TMS device may only function on a single brain area position. In other words, if different brain areas are about to be stimulated, relevant personnel need to move the TMS device to the corresponding positions. Moreover, when performing treatment of co-morbidities in multiple brain areas, relevant personnel still have to replace the TMS device with coils of different configurations.

Therefore, the existing TMS device is not convenient in use.

### SUMMARY

The disclosure is directed to a driving method of a transcranial magnetic stimulation 3D coil device, which is adapted to solve the above technical problem.

An embodiment of the disclosure provides a driving method of a transcranial magnetic stimulation 3D coil device, which is adapted to a driving device, where the transcranial magnetic stimulation 3D coil device includes multiple coils, and central axes of each of the coils are perpendicular to each other. The method includes the following. A first signal peak and a second signal peak corresponding to a specified rotating magnetic field direction are determined. The first signal peak and the second signal peak respectively correspond to a first coil and a second coil in the coils. A first pulse current having the first signal peak is provided to the first coil. The first pulse current signal stimulates the first coil to provide a first magnetic field. A second pulse current signal having the second signal peak is provided to the second coil. The second pulse current signal stimulates the second coil to provide a second magnetic field, and the first magnetic field and the second magnetic field form a rotating magnetic field having the specified rotating magnetic field direction.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1A to FIG. 1D are different views of a transcranial magnetic stimulation 3D coil device according to an embodiment of the disclosure.

FIG. 2 is a schematic diagram of different transcranial magnetic stimulation electric fields illustrated according to FIG. 1.

FIG. 3A and FIG. 3B are different views of a transcranial magnetic stimulation 3D coil device according to an embodiment of the disclosure.

FIG. 4 is a schematic diagram of the transcranial magnetic stimulation 3D coil device shown in FIG. 3A and FIG. 3B.

FIG. 5 is a schematic diagram of a transcranial magnetic stimulation 3D coil device driven by a driving device according to an embodiment of the disclosure.

FIG. 6 is a flowchart of a driving method of a transcranial magnetic stimulation 3D coil device according to an embodiment of the disclosure.

FIG. 7 is a waveform diagram drawn according to Table 1.

FIG. 8 is a schematic diagram of first and second pulse current signals shown in Table 1.

FIG. 9 is an application scenario diagram according to an embodiment of the disclosure.

FIG. 10 is an application scenario diagram according to an embodiment of the disclosure.

FIG. 11 is an application scenario diagram according to an embodiment of the disclosure.

### DESCRIPTION OF THE EMBODIMENTS

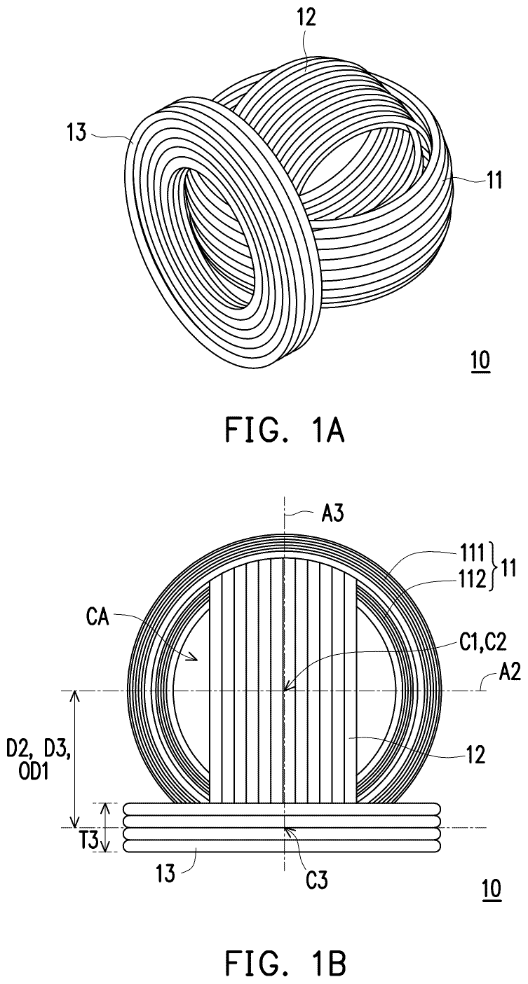

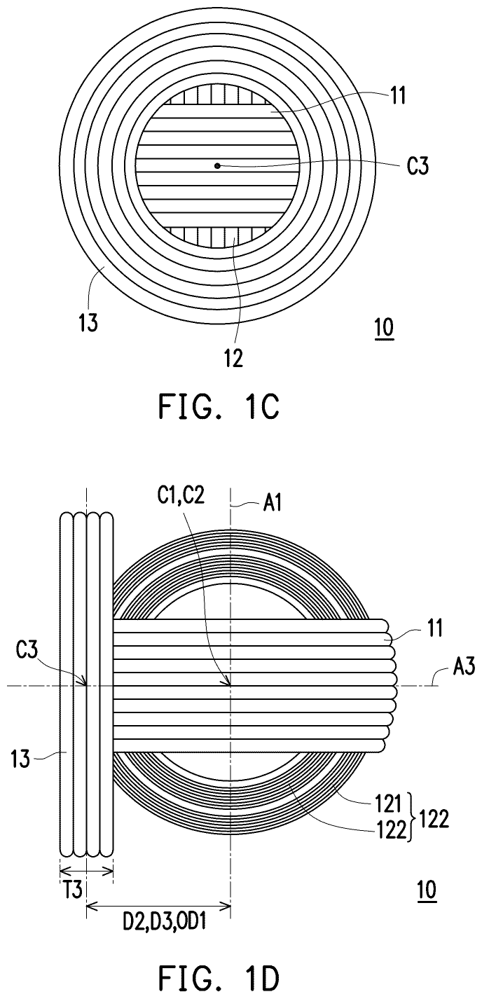

Referring to FIG. 1A to FIG. 1D, which are different views of a transcranial magnetic stimulation 3D coil device according to an embodiment of the disclosure.

In FIG. 1A to FIG. 1D, a transcranial magnetic stimulation 3D coil device 10 includes a first coil 11, a second coil 12 and a third coil 13. The first coil 11 has a first central axis A1 and a central area CA. The second coil 12 has a second central axis A2, and the second coil 12 is located in the central area CA. The third coil 13 has a third central axis A3 and is located on one side of the first coil 11, where the first central axis Al, the second central axis A2 and the third central axis A3 are perpendicular to each other.

In FIG. 1B, the first coil 11 has an outer sub-coil 111 and an inner sub-coil 112, and the second coil 12 is between the outer sub-coil 111 and the inner sub-coil 112. In addition, in FIG. 1D, the second coil 12 has an outer sub-coil 121 and an inner sub-coil 122, and the outer sub-coil 121 of the second coil 12 is between the outer sub-coil 111 and the inner sub-coil 112, and the inner sub-coil 112 of the first coil 11 is between the outer sub-coil 121 and the inner sub-coil 122 of the second coil 12, but the disclosure is not limited thereto.

In an embodiment, the first coil 11 has a first center point C1, the second coil 12 has a second center point C2, and a first distance between the first center point C1 and the second center point C2 is not greater than a first threshold value. In FIG. 1B and FIG. 1D, the first center point C1 may be, for example, coincided with/the same as the second center point C2. In different embodiments, the first threshold value may be set to any value that may be considered to make the first center point C1 sufficiently close to the second center point C2 according to the designer's needs, but the disclosure is not limited thereto.

In an embodiment, the third coil 13 has a third center point C3, and a second distance D2 between the third center point C3 and the first center point C1 or a third distance D3 between the third center point C3 and the second center point C2 is within a preset range, where the preset range is determined based on an outer diameter OD1 of the first coil 11 and a thickness T3 of the third coil 13.

In an embodiment, an upper limit of the preset range is, for example, a sum of the outer diameter OD1 and a half of the thickness T3, and a lower limit of the preset range is, for example, the outer diameter OD1 minus a half of the thickness T3, but the disclosure is not limited thereto.

In the situation of FIG. 1A to FIG. 1D, since the first center point C1 is assumed to be coincided with the second center point C2, the second distance D2 between the third center point C3 and the first center point C1 may be equal to the third distance D3 between the third center point C3 and the second center point C2, but the disclosure is not limited thereto.

In addition, in the situation of FIG. 1A to FIG. 1D, it is assumed that the outer diameter OD1 is equal to the second distance D2 (and the third distance D3), but the disclosure is not limited thereto.

In an embodiment, a number of layers of the first coil 11 is 2, and a number of turns of each layer of the first coil 11 is 10 turns or 8 turns.

In an embodiment, a number of layers of the second coil 12 is 2, and a number of turns of each layer of the second coil 12 is 12 turns or 10 turns.

In an embodiment, a number of layers of the third coil 13 is 4, and a number of turns of each layer of the third coil 13 is 6 turns, 5 turns, 4 turns or 3 turns.

In an embodiment, a coil diameter of at least one of the first coil 11, the second coil 12 and the third coil 13 is less than 3.5 mm.

In an embodiment, a coil resistance of at least one of the first coil 11, the second coil 12 and the third coil 13 is less than 45 mQ.

In an embodiment, a coil inductance of at least one of the first coil 11, the second coil 12 and the third coil 13 is less than uH.

In an embodiment, at least one of the first coil 11, the second coil 12 and the third coil 13 is made of Ritz wire.

In an embodiment, a wire diameter of at least one of the first coil 11, the second coil 12 and the third coil 13 is 30 American wire gauge (AWG).

In an embodiment, the first coil 11 is stimulated to provide a first electric field, the second coil 12 is stimulated to provide a second electric field, the third coil 13 is stimulated to provide a third electric field, and the first electric field, the second electric field and the third electric field cooperate to form a transcranial magnetic stimulation electric field corresponding to the transcranial magnetic stimulation 3D coil device 10.

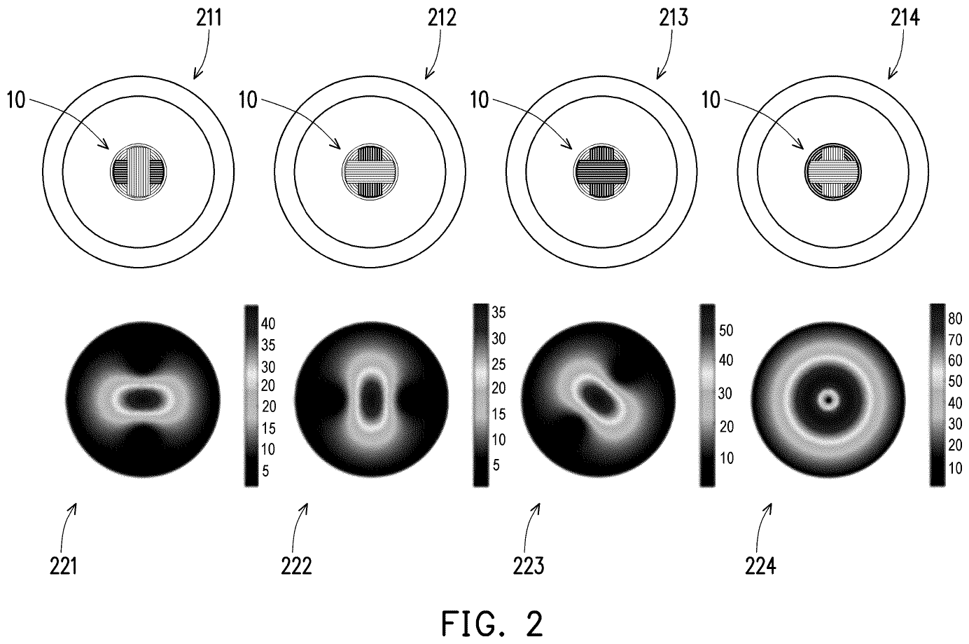

Referring to FIG. 2, FIG. 2 is a schematic diagram of different transcranial magnetic

stimulation electric fields illustrated according to FIG. 1. In FIG. 2, a scenario 211 is, for example, a scenario in which only the first coil 11 of the transcranial magnetic stimulation 3D coil device 10 is stimulated to provide the first electric field. Since the second coil 12 and the third coil 13 are not stimulated, a transcranial magnetic stimulation electric field 221 provided by the transcranial magnetic stimulation 3D coil device 10 is only formed by the first electric field of the first coil 11.

In addition, a scenario 212 is, for example, a scenario in which only the second coil 12 of the transcranial magnetic stimulation 3D coil device 10 is stimulated to provide the second electric field. Since the first coil 11 and the third coil 13 are not stimulated, a transcranial magnetic stimulation electric field 222 provided by the transcranial magnetic stimulation 3D coil device 10 is only formed by the second electric field of the second coil 12.

A scenario 213 is, for example, a scenario in which the first coil 11 and the second coil 12 of the transcranial magnetic stimulation 3D coil device 10 are simultaneously stimulated to respectively provide the first electric field and the second electric field. Since the third coil 13 is not stimulated, a transcranial magnetic stimulation electric field 223 provided by the transcranial magnetic stimulation 3D coil device 10 is cooperatively formed by the first electric field of the first coil 11 and the second electric field of the second coil 12.

It may be seen from FIG. 2, by stimulating the first coil 11 and the second coil 12 at the same time, the transcranial magnetic stimulation electric field 223 may be rotated accordingly. In this case, the transcranial magnetic stimulation electric field 223 may be easily rotated according to the designer's needs by adjusting a current intensity used to stimulate the first coil 11 and/or the second coil 12.

A scenario 214 is, for example, a scenario in which only the third coil 13 of the transcranial magnetic stimulation 3D coil device 10 is stimulated to provide the third electric field. Since the first coil 11 and the second coil 12 are not stimulated, a transcranial magnetic stimulation electric field 224 provided by the transcranial magnetic stimulation 3D coil device 10 is only formed by the third electric field of the third coil 13.

It may be seen from the transcranial magnetic stimulation electric fields 223 and 224 that if the third coil 13 is further stimulated in the case that the first coil 11 and the second coil 12 are already stimulated, the transcranial magnetic stimulation electric field 223 may be accordingly shifted left/right.

Therefore, by simply adjusting the current intensity used to stimulate the first coil 11, the second coil 12 and/or the third coil 13, the transcranial magnetic stimulation electric field 223 may present corresponding rotation and shift according to the designer's needs, so that the formed transcranial magnetic stimulation electric field may present a pattern required by the designer. In this way, the transcranial magnetic stimulation 3D coil device 10 of the disclosure may treat different brain areas of a patient through the transcranial magnetic stimulation electric field with different patterns without replacing the coils.

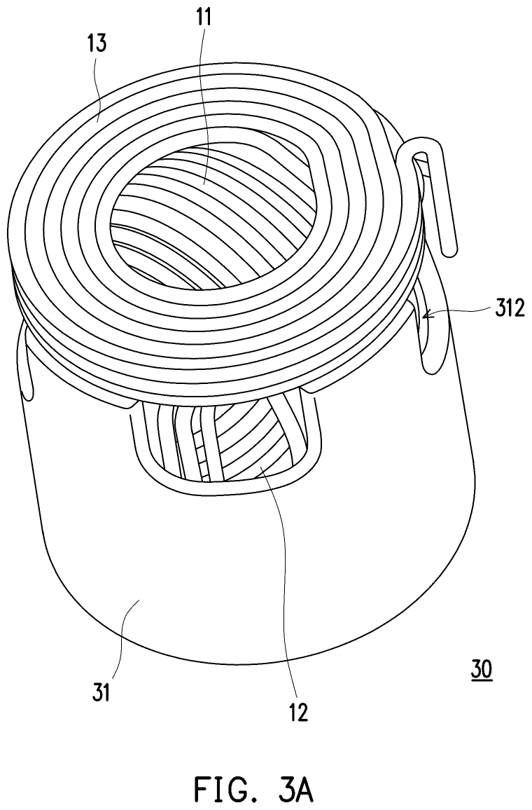

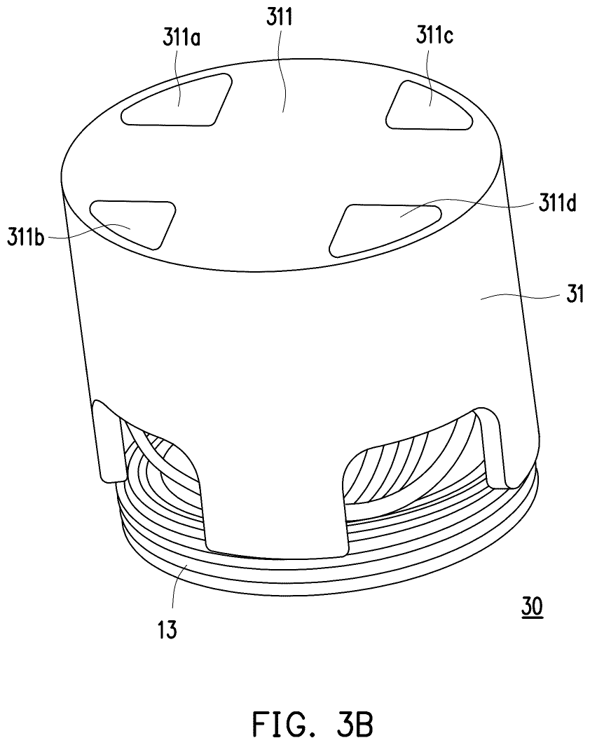

Referring to FIG. 3A and FIG. 3B, which are different views of a transcranial magnetic stimulation 3D coil device according to an embodiment of the disclosure. In the embodiment, in addition to the various components of the transcranial magnetic stimulation 3D coil device 10 in FIG. 1A to FIG. 1D (for example, the first coil 11, the second coil 12 and the third coil 13), a transcranial magnetic stimulation 3D coil device 30 further includes a cylindrical housing 31 for accommodating the first coil 11 and the second coil 12.

In FIG. 3, the cylindrical housing 31 has a bottom surface 311 and an opening 312 relative to the bottom surface, where the bottom surface 311 is provided with ventilation holes 311a-311d, and the opening 312 is used to sleeve the first coil 11 and the second coil 12.

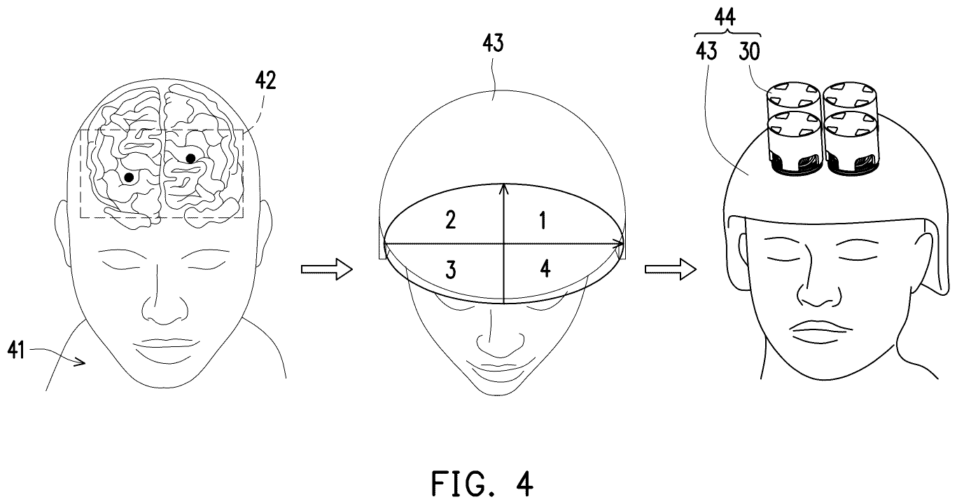

Referring to FIG. 4, FIG. 4 is a schematic diagram of the transcranial magnetic stimulation 3D coil device shown in FIG. 3A and FIG. 3B.

In FIG. 4, it is assumed that a brain area 42 of a patient 41 is determined/diagnosed to require treatment via TMS technology, relevant personnel (such as doctors, etc.) may plan specified positions for setting one or more transcranial magnetic stimulation 3D coils 30 on a wearable device 43 (such as a helmet to be worn by the patient 41), for example, the positions marked with numbers 1 to 4 in FIG. 4, but the disclosure is not limited thereto.

In this way, several transcranial magnetic stimulation 3D coils 30 may be arranged at the corresponding specified positions. Then, each transcranial magnetic stimulation 3D coil device 30 is stimulated to provide a transcranial magnetic stimulation electric field, and the transcranial magnetic stimulation electric fields corresponding to each of the transcranial magnetic stimulation 3D coil devices 30 cooperate to form an integrated transcranial magnetic stimulation electric field corresponding to a transcranial magnetic stimulation 3D coil device 44 (which includes the wearable device 43 and one or more transcranial magnetic stimulation 3D coil devices 30).

In this way, the transcranial magnetic stimulation 3D coil device 44 of the disclosure may treat different brain areas of the patient through the integrated transcranial magnetic stimulation electric field with different patterns without changing the coils.

From the above, it is known that the embodiments of the disclosure provide a transcranial magnetic stimulation 3D coil device with a novel structure, which includes three coils with central axes perpendicular to each other. In this case, by simply adjusting the current intensity used to stimulate the above first coils, the transcranial magnetic stimulation electric field may present corresponding rotation and shift according to the designer's needs, thereby enabling the transcranial magnetic stimulation electric field provided by the transcranial magnetic stimulation 3D coil device to present the pattern required by the designer. In this way, the transcranial magnetic stimulation 3D coil device of the disclosure may treat different brain areas of the patient through the transcranial magnetic stimulation electric field with different patterns without replacing the coils.

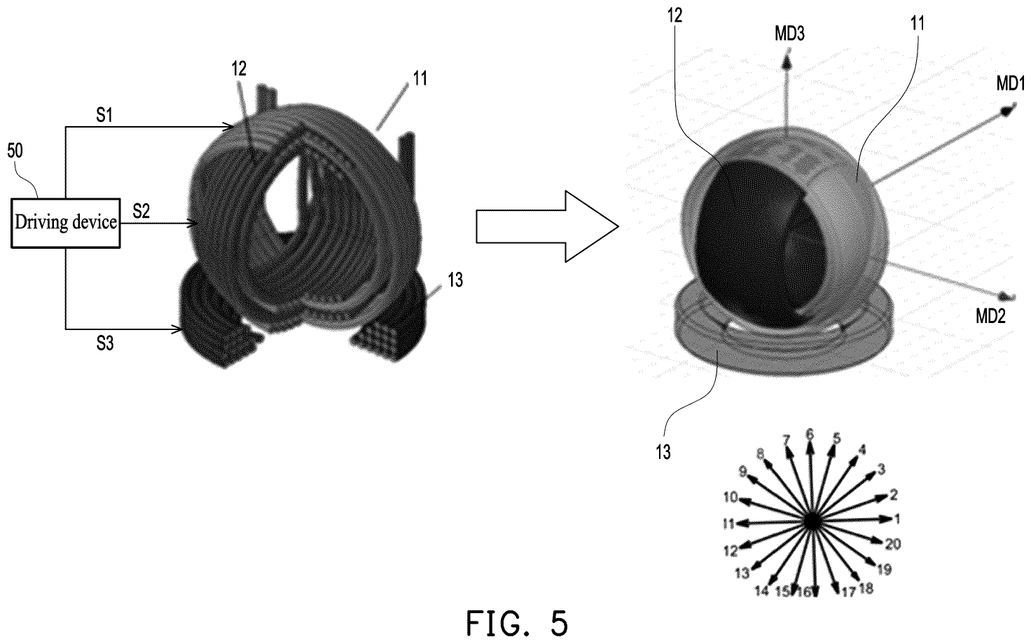

Referring to FIG. 5, FIG. 5 is a schematic diagram of a transcranial magnetic stimulation 3D coil device driven by a driving device according to an embodiment of the disclosure.

In FIG. 5, a driving device 50 may respectively provide a first pulse current signal S1, a second pulse current signal S2 and a third pulse current signal S3 to the first coil 11, the second coil 12 and the third coil 13. In this case, the first coil 11, the second coil 12 and the third coil 13 may be respectively stimulated to generate corresponding magnetic fields according to the Ampere right-hand rule.

In FIG. 5, it is assumed that a first magnetic field generated by the first coil 11 after being stimulated by the first pulse current signal S1 has a first magnetic field direction MD1, and a second magnetic field generated by the second coil 12 after being stimulated by the second pulse current signal S2 has a second magnetic field direction MD2, and a third magnetic field generated by the third coil 13 after being stimulated by the third pulse current signal S3 has a third magnetic field direction MD3, but the disclosure is not limited thereto.

For the ease of understanding, the first magnetic field direction MD1, the second magnetic field direction MD2 and the third magnetic field direction MD3 may be respectively understood as a +Y direction, a +X direction and a +Z direction in a 3D space shown in FIG. 5, but the disclosure is not limited thereto.

In an embodiment of the disclosure, the driving device 50 may control a rotation direction and/or a shift position of a magnetic field formed by the first, second, and third magnetic fields by regulating the first pulse current signal S1, the second pulse current signal S2, and/or the third pulse current signal S3, which is further described below.

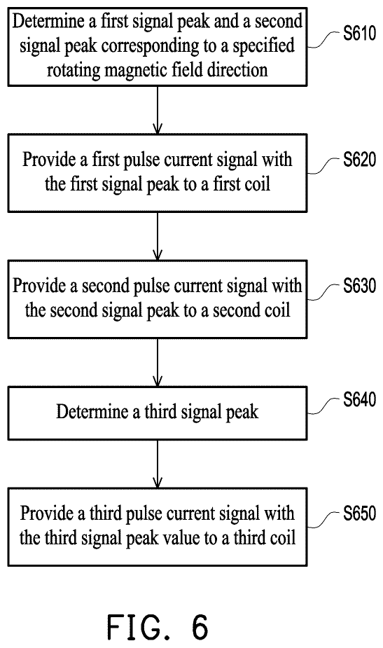

Referring to FIG. 6, FIG. 6 is a flowchart of a driving method of a transcranial magnetic stimulation 3D coil device according to an embodiment of the disclosure. The driving method of the embodiment may be executed by the driving device 50 of FIG. 5, and details of each step in FIG. 6 will be described below with reference to the components shown in FIG. 5.

In step S610, the driving device 50 determines a first signal peak and a second signal peak corresponding to a specified rotating magnetic field direction, where the first signal peak and the second signal peak respectively correspond to the first coil 11 and the second coil 12.

In the embodiment of the disclosure, the specified rotating magnetic field direction is, for example, a direction that an operator wants an N pole of a rotating magnetic field formed by the first and second magnetic fields to point, but the disclosure is not limited thereto.

In the embodiment of the disclosure, the operator may, for example, select a desired one from preset K candidate rotating magnetic field directions as the specified rotating magnetic field direction. In FIG. 5, the specified rotating magnetic field direction is, for example, one of 20 (i.e., K is 20) candidate rotating magnetic field directions shown in a lower right corner of FIG. 5, where the direction of number 1, for example, corresponds to the +X direction (i.e., corresponds to the second magnetic field direction MD2), and the direction of number 6, for example, corresponds to the +Y direction (i.e., corresponds to the first magnetic field direction MD1) but the disclosure may not be limited thereto.

In the embodiment of the disclosure, each candidate rotating magnetic field direction may have a corresponding first reference signal peak and a second reference signal peak, where the first reference signal peak and the second reference signal peak corresponding to a j^{th} candidate rotating magnetic field direction among the K candidate rotating magnetic field directions may be represented as I_{1,j}(A) and I_{2,j}(A).

In the embodiment of the disclosure, the first reference signal peak and the second reference signal peak of each candidate rotating magnetic field direction also correspond to the first coil 11 and the second coil 12 respectively.

In the scenario of FIG. 6, the first reference signal peak and the second reference signal peak corresponding to the 20 candidate rotating magnetic field directions (numbered from 1 to 20) are shown in Table 1 below.

TABLE 1

| | | Second reference | First reference |
|---|---|---|---|
| | | signal peak | signal peak |
| No. | Angle | (I_{2, j}(A)) | (I_{1, j}(A)) |
| 1 | 0 | 7881.05 | 0.00 |
| 2 | 18 | 7447.59 | 2648.34 |
| 3 | 36 | 6218.15 | 5003.33 |
| 4 | 54 | 4303.05 | 6820.50 |
| 5 | 72 | 1930.86 | 7896.16 |
| 6 | 90 | 0.00 | 8148.75 |
| 7 | 108 | −1930.86 | 7896.14 |
| 8 | 126 | −4303.05 | 6820.50 |
| 9 | 144 | −6218.15 | 5003.33 |
| 10 | 162 | −7447.59 | 2648.34 |
| 11 | 180 | −7881.05 | 0 |
| 12 | 198 | −7447.59 | −2648.34 |
| 13 | 216 | −6218.15 | −5003.33 |
| 14 | 234 | −4303.05 | −6820.50 |
| 15 | 252 | −1930.86 | −7896.16 |
| 16 | 270 | 0 | −8148.75 |
| 17 | 288 | 1930.86 | −7896.14 |
| 18 | 306 | 4303.05 | −6820.50 |
| 19 | 324 | 6218.15 | −5003.33 |
| 20 | 342 | 7447.59 | −2648.34 |

Based on the above, in an embodiment, the operator may, for example, select a desired one from the 20 candidate rotating magnetic field directions in Table 1 as the above-mentioned specified rotating magnetic field direction, and use the corresponding first reference signal peak and second reference signal peak (with a unit of Ampere (A)) as the first signal peak and the second signal peak in step S610.

For example, it is assumed that the operator wants to make the N pole of the rotating magnetic field formed by the first and second magnetic fields pointing in the direction corresponding to number 4 (i.e., the direction of number 4 is used as the specified rotating magnetic field direction), then the driving device 50 may, for example, respectively use 6820.50 and 4303.05 as the first signal peak and the second signal peak considered in step S610.

For another embodiment, it is assumed that the operator wants to make the N pole of the rotating magnetic field formed by the first and second magnetic fields pointing in the direction corresponding to number 19 (that is, the direction of number 19 is used as the specified rotating magnetic field direction), then the driving device 50 may, for example, respectively use −5003.33 and 6218.15 as the first signal peak and the second signal peak considered in step S610.

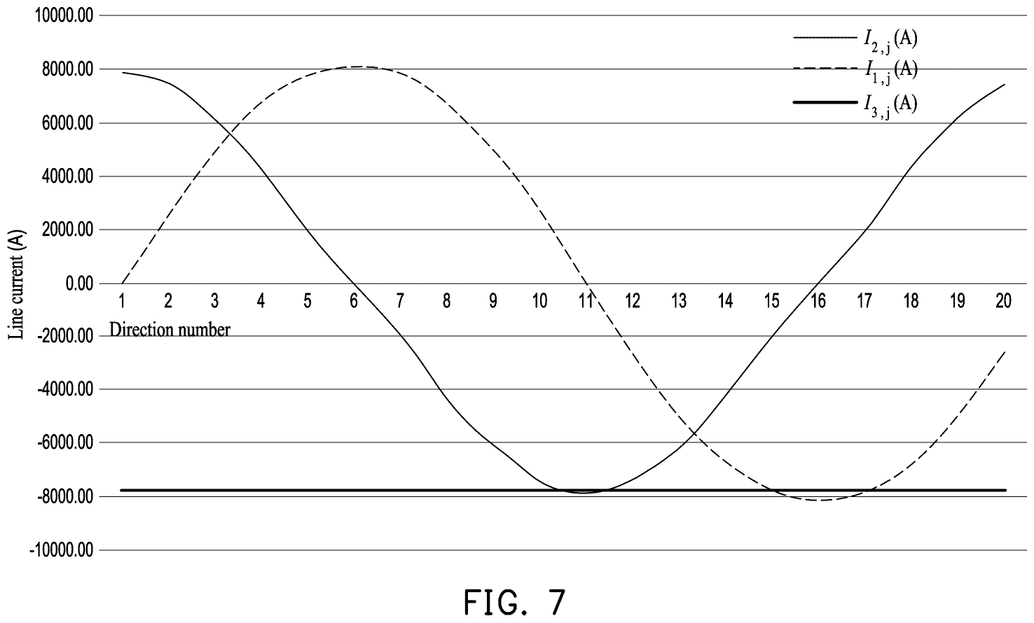

In an embodiment, the content of Table 1 may be plotted as waveforms as shown in FIG. 7. It can be seen from FIG. 7 that the changes in the first reference signal peak corresponding to different direction numbers have a trend corresponding to a first sinusoidal wave (such as a sine wave), while the changes in the second reference signal peak corresponding to different direction numbers have a trend corresponding to a second sinusoidal wave (such as a cosine wave).

In some embodiments,

$$- 1 \leq \frac{{I}_{1 , j} ( A )}{{I}_{1 , max} ( A )} \leq 1 , - 1 \leq \frac{{I}_{2 , j} ( A )}{{I}_{2 , max} ( A )} \leq 1 ,$$

where I_{1,max}(A) is the maximum value in the first reference signal peaks corresponding to each of the candidate rotating magnetic field directions, I_{2,max}(A) is the maximum value in the second reference signal peaks corresponding to each of the candidate rotating magnetic field directions. In addition, in some embodiments,

$$- 1.414 \leq \frac{{I}_{2 , j} ( A )}{{I}_{2 , max} ( A )} + \frac{{I}_{1 , j} ( A )}{{I}_{1 , max} ( A )} \leq 1.414 .$$

In the scenario of Table 1, I_{1,max}(A) is 8148.75, while I_{2,max}(A) is 7881.05. Based on this, Table 1 may be further extended into Table 2 below.

TABLE 2

| No. | Angle | I_{2,j}(A) | $\frac{{I}_{2 , j} ( A )}{{I}_{2 , max} ( A )}$ | I_{1,j}(A) | $\frac{{I}_{1 , j} ( A )}{{I}_{1 , max} ( A )}$ | $\frac{{I}_{2 , j} ( A )}{{I}_{2 , max} ( A )} + \frac{{I}_{1 , j} ( A )}{{I}_{1 , max} ( A )}$ |
|---|---|---|---|---|---|---|
| 1 | 0 | 7881.05 | 1.00 | 0.00 | 0.00 | 1.00 |
| 2 | 18 | 7447.59 | 0.94 | 2648.34 | 0.32 | 1.27 |
| 3 | 36 | 6218.15 | 0.79 | 5003.33 | 0.61 | 1.40 |
| 4 | 54 | 4303.05 | 0.55 | 6820.50 | 0.84 | 1.38 |
| 5 | 72 | 1930.86 | 0.25 | 7896.16 | 0.97 | 1.21 |
| 6 | 90 | 0.00 | 0.00 | 8148.75 | 1.00 | 1.00 |
| 7 | 108 | −1930.86 | −0.25 | 7896.14 | 0.97 | 0.72 |
| 8 | 126 | −4303.05 | −0.55 | 6820.50 | 0.84 | 0.29 |
| 9 | 144 | −6218.15 | −0.79 | 5003.33 | 0.61 | −0.18 |
| 10 | 162 | −7447.59 | −0.94 | 2648.34 | 0.32 | −0.62 |
| 11 | 180 | −7881.05 | −1.00 | 0 | 0.00 | −1.00 |
| 12 | 198 | −7447.59 | −0.94 | −2648.34 | −0.32 | −1.27 |
| 13 | 216 | −6218.15 | −0.79 | −5003.33 | −0.61 | −1.40 |
| 14 | 234 | −4303.05 | −0.55 | −6820.50 | −0.84 | −1.38 |
| 15 | 252 | −1930.86 | −0.25 | −7896.16 | −0.97 | −1.21 |
| 16 | 270 | 0 | 0.00 | −8148.75 | −1.00 | −1.00 |
| 17 | 288 | 1930.86 | 0.25 | −7896.14 | −0.97 | −0.72 |
| 18 | 306 | 4303.05 | 0.55 | −6820.50 | −0.84 | −0.29 |
| 19 | 324 | 6218.15 | 0.79 | −5003.33 | −0.61 | 0.18 |
| 20 | 342 | 7447.59 | 0.94 | −2648.34 | −0.32 | 0.62 |

In addition, in the scenario of FIG. 7, the third (reference) signal peak corresponding to the third coil 13 may be designed to a constant value (for example, −8000 ampere as shown) to avoid shifting of the rotating magnetic field formed by the first and second magnetic fields, but the disclosure may not be limited thereto.

Referring to FIG. 6 again, after determining the required first and second signal peaks, the driving device 50 may continue to execute step S620 to provide the first pulse current signal S1 with the first signal peak to the first coil 11, and execute step S630 to provide the second pulse current signal S2 with the second signal peak to the second coil 12. In the embodiment of the 10 disclosure, the first pulse current signal S1 stimulates the first coil 11 to provide the first magnetic field, the second pulse current signal S2 stimulates the second coil 12 to provide the second magnetic field, and the first magnetic field and the second magnetic field form a rotating magnetic field with a specified rotating magnetic field direction.

In other embodiments, the order of steps S620 and S630 may be reversed or executed at the same time, but the disclosure is not limited thereto.

In different embodiments, the first pulse current signal S1 and the second pulse current signal S2 may individually be a single-phase pulse current signal or a biphasic pulse current signal.

In some embodiments, a first current change rate of the first pulse current signal S1 is, for example, 13.67 A/ms to 136.67 A/ms. In addition, a second current change rate of the second pulse current signal S2 is, for example, 12.89 A/ms to 128.93 A/ms, but the disclosure is not limited thereto.

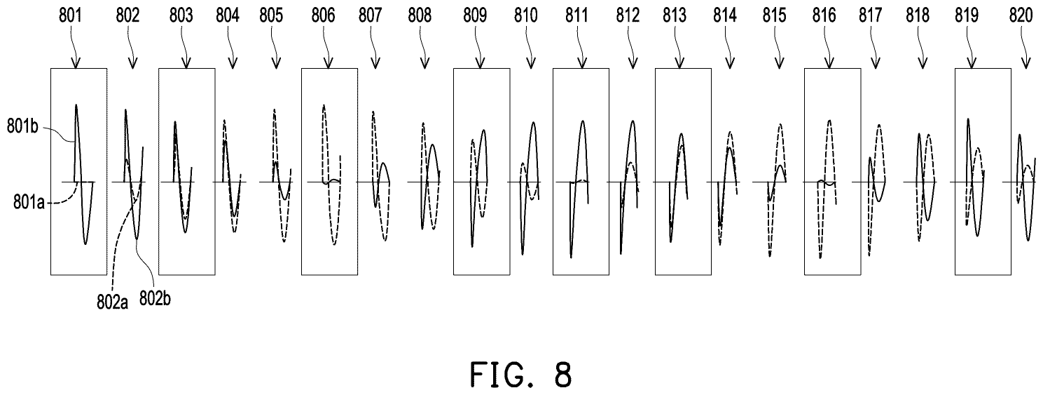

Referring to FIG. 8, FIG. 8 is a schematic diagram of the first and second pulse current signals shown in Table 1. In each of waveform diagrams 801 to 820 shown in FIG. 8, the corresponding first and second pulse current signals are, for example, biphasic pulse current signals (i.e., pulse current signals including positive half cycles and negative half cycles), and the waveform diagrams 801 to 820 may respectively correspond to the 20 candidate rotating magnetic field directions in Table 1. In other embodiments, the first and second pulse current signals may also be single-phase pulse current signals (i.e., pulse current signals including only positive half cycles or negative half cycles).

In each of the waveform diagrams 801 to 820, a waveform corresponding to the first pulse current signal S1 is shown as a dotted line, and a waveform corresponding to the second pulse current signal S2 is shown as a solid line.

For example, in Table 1, the first reference signal peak and the second reference signal peak corresponding to the candidate rotating magnetic field direction of number 1 are 0.00 and 7881.05 respectively. Based on the above, in the waveform diagram 801 of FIG. 8 (which corresponds to the candidate rotating magnetic field direction of number 1), a first reference signal peak of a first pulse current signal 801a is, for example, 0.00, and a second reference signal peak of a second pulse current signal 801b is, for example, 7881.05. In other words, if the operator wants to make the N pole of the rotating magnetic field formed by the first and second magnetic fields pointing in the direction corresponding to number 1 (i.e., the direction of number 1 is used as the specified rotating magnetic field direction), the driving device 50 may, for example, provide the first pulse current signal 801a to the first coil 11 in step S620, and provide the second pulse current signal 801b to the second coil 12 in step S630.

For another example, in Table 1, the first reference signal peak and the second reference signal peak corresponding to the candidate rotating magnetic field direction of number are 2648.34 and 7447.59 respectively. Based on the above, in the waveform diagram 802 of FIG. 8 (which corresponds to the candidate rotating magnetic field direction of number 2), a first reference signal peak of a first pulse current signal 802a is, for example, 2648.34, and a second reference signal peak of a second pulse current signal 802b is, for example, 7447.59. In other words, if the operator wants to make the N pole of the rotating magnetic field formed by the first and second magnetic fields pointing in the direction corresponding to number 2 (i.e., the direction of number 2 is used as the specified rotating magnetic field direction), the driving device 50 may, for example, provide the first pulse current signal 802a to the first coil 11 in step S620, and provide the second pulse current signal 802b to the second coil 12 in step S630.

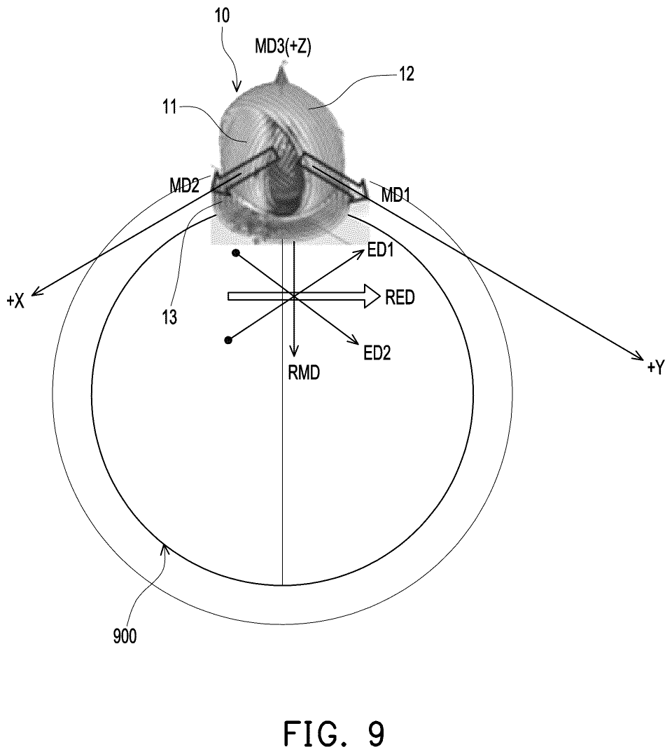

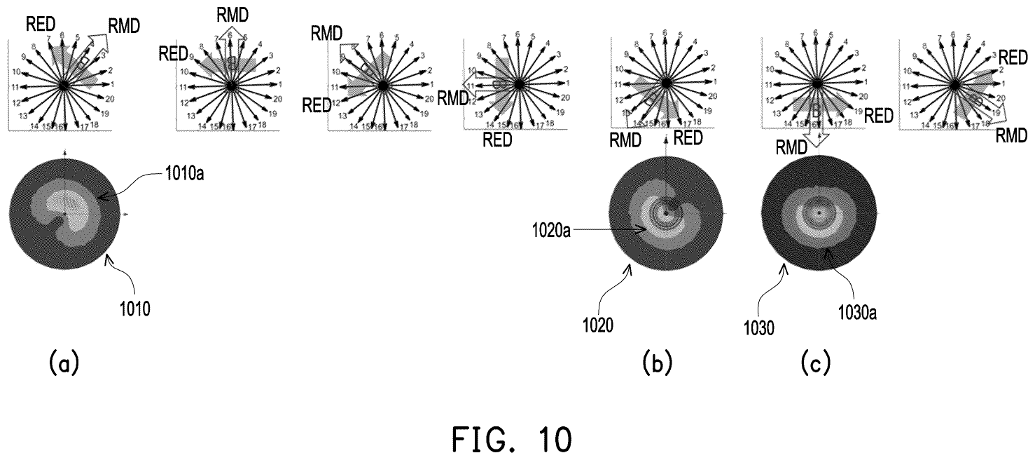

To facilitate understanding, FIG. 9 is used for further explanation below.

Referring to FIG. 9, FIG. 9 is an application scenario diagram according to an embodiment of the disclosure. In FIG. 9, the transcranial magnetic stimulation 3D coil device 10 may be, for example, disposed on a model 900 for simulating a human head. The model 900 is, for example, a homogeneous sphere with a radius of 8.5 cm and an isotropic conductivity of 0.33 Sm. In addition, a distance between a cortical surface and a gray matter-white matter interface of the model 900 is 1.5 cm to 2 cm.

In FIG. 9, it is assumed that the direction of number 4 in Table 1 is selected as the specified rotating magnetic field direction in step S610. Correspondingly, the driving device 50 may, for example, determine the first pulse current signal S1 and the second pulse current signal S1 used to drive the first coil 11 and the second coil 12 according to the waveform diagram 804 in FIG. 8 and the relevant information corresponding to the number 4 in Table 1.

In FIG. 9, the first coil 11 may, for example, provide a first magnetic field with the first magnetic field direction MD1 after being stimulated by the first pulse current signal S1, and the first magnetic field may correspondingly generate a first electric field with a first electric field direction ED1 (which is orthogonal to the first magnetic field direction MD1) in the model 900. In addition, the second coil 12 may provide a second magnetic field with the second magnetic field direction MD2 after being stimulated by the second pulse current signal S2, and the second magnetic field may correspondingly generate a second electric field with a second electric field direction ED2 (which is orthogonal to the second magnetic field direction MD2) in the model 900.

In this case, the first electric field and the second electric field may be superimposed in the model 900 accordingly to form a superimposed electric field with an integrated electric field direction RED.

From another point of view, the first magnetic field and the second magnetic field may be superimposed to form a rotating magnetic field with a specified rotating magnetic field direction RMD (for example, the direction of number 4 in Table 1), and this rotating magnetic field may correspondingly form a superimposed electric field with the integrated electric field direction RED (which is orthogonal to the specified rotating magnetic field direction RMD) in the model 900.

It may be seen from the above, through the method provided by the disclosure, the operator may adjust the superimposed electric field formed in the model 900 by the first coil 11 and the second coil 12 by adjusting the first and second pulse current signals without moving a position of the transcranial magnetic stimulation 3D coil device 10. In this way, when the transcranial magnetic stimulation 3D coil device 10 is placed on the patient's head, the electric field with a specific direction may be more flexibly applied to the brain area to be treated, thereby improving the convenience of treatment.

In some embodiments, in addition to driving the first coil 11 and the second coil 12 in the above manner, in the method of the disclosure, the third coil 13 may be further driven in a specific manner, so that the first coil 11, the second coil 12 and the third coil 13 may generate a magnetic field/electric field with a desired shape/direction after being driven.

Referring to FIG. 6 again, in an embodiment, the driving device 50 may further perform step S640 to determine a third signal peak, where the third signal peak corresponds to the third coil 13.

Then, in step S650, the driving device 50 provides the third pulse current signal S3 with the third signal peak value to the third coil 13.

In an embodiment of the disclosure, the third pulse current signal S3 may be a single-phase pulse current signal or a biphasic pulse current signal. In addition, a third current change rate of the third pulse current signal S3 is, for example, 12.53 A/ms to 125.33 A/ms, but the disclosure is not limited thereto.

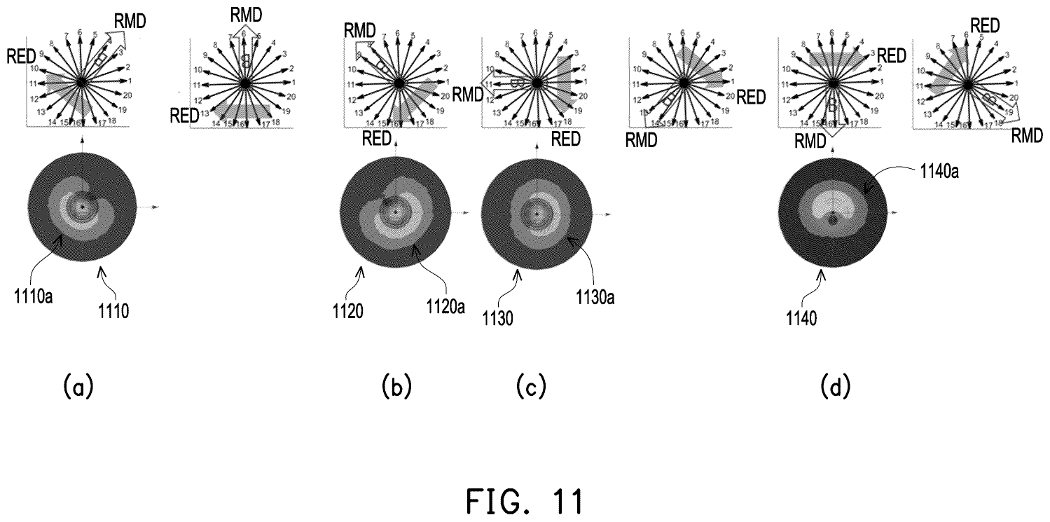

In order to make the above concepts easier to understand, FIG. 10 and FIG. 11 will be used for further explanation below.

Referring to FIG. 10, FIG. 10 is an application scenario diagram according to an embodiment of the disclosure.

In each scenario of FIG. 10, it is assumed that the driving device 50 drives the third coil 13 with the third pulse current signal S3 having the third signal peak while driving the first coil 11 and the second coil 12. In the embodiment, it is assumed that the third coil 13 is driven to provide a third magnetic field of the third magnetic field direction MD3 corresponding to the +Z direction. In this case, the third magnetic field will correspondingly affect the rotating magnetic field formed by the first and second magnetic fields. In other words, the third electric field induced by the third magnetic field will also affect the superimposed electric field formed by the first and second electric fields.

For example, in scenario (a) of FIG. 10, it is assumed that the specified rotating magnetic field direction is the direction of number 4 in Table 1, the driving device 50 may, for example, drive the first coil 11 and the second coil 12 according to the previous teaching to generate a rotating magnetic field with the specified rotating magnetic field direction RMD (such as the direction of number 4 in Table 1), and this rotating magnetic field may correspondingly form a superimposed electric field with the integrated electric field direction RED.

In addition, when the third coil 13 is also driven, the third magnetic field formed by the third coil 13 may cause the rotating magnetic field formed by the first and second magnetic fields to shift toward a specified magnetic field shift position, and the shifted rotating magnetic field (which may be understood as a superimposed magnetic field formed by superimposing the first, second, and third magnetic fields) may correspondingly induce an electric field shown as an electric field diagram 1010.

In the electric field diagram 1010, the electric field shown may be understood as the superimposed electric field of the first and second electric fields after being affected by the third electric field, and may also be understood as a specific superimposed electric field formed by superimposing the first, second, and third electric fields. From the electric field diagram 1010, it may be seen that the specific superimposed electric field shown is shifted towards a specific electric field position 1010a.

For another example, in scenario (b) of FIG. 10, it is assumed that the specified rotating magnetic field direction is the direction of number 14 in Table 1, the driving device 50 may drive the first coil 11 and the second coil 12 according to the previous teaching to generate a rotating magnetic field with the specified rotating magnetic field direction RMD (for example, the direction of number 14 in Table 1), and this rotating magnetic field may correspondingly form a superimposed electric field with the integrated electric field direction RED.

In addition, when the third coil 13 is also driven, the third magnetic field formed by the third coil 13 may cause the rotating magnetic field formed by the first and second magnetic fields to shift toward the specified magnetic field shift position, and the shifted rotating magnetic field (which may be understood as a superimposed magnetic field formed by superimposing the first, second, and third magnetic fields) may correspondingly induce an electric field shown as an electric field diagram 1020.

In the electric field diagram 1020, the electric field shown may be understood as the superimposed electric field of the first and second electric fields after being affected by the third electric field, and may also be understood as a specific superimposed electric field formed by superimposing the first, second, and third electric fields. From the electric field diagram 1020, it may be seen that the specific superimposed electric field shown is shifted towards a specific electric field position 1020a.

For another example, in scenario (c) of FIG. 10, it is assumed that the specified rotating magnetic field direction is the direction of number 16 in Table 1, the driving device 50 may drive the first coil 11 and the second coil 12 according to the previous teaching to generate a rotating magnetic field with the specified rotating magnetic field direction RMD (for example, the direction of number 16 in Table 1), and this rotating magnetic field may correspondingly form a superimposed electric field with the integrated electric field direction RED.

In addition, when the third coil 13 is also driven, the third magnetic field formed by the third coil 13 may cause the rotating magnetic field formed by the first and second magnetic fields to shift toward the specified magnetic field shift position, and the shifted rotating magnetic field (which may be understood as a superimposed magnetic field formed by superimposing the first, second, and third magnetic fields) may correspondingly induce an electric field shown as an electric field diagram 1030.

In the electric field diagram 1030, the electric field shown may be understood as the superimposed electric field of the first and second electric fields after being affected by the third electric field, and may also be understood as a specific superimposed electric field formed by superimposing the first, second, and third electric fields. From the electric field diagram 1030, it may be seen that the specific superimposed electric field shown is shifted towards a specific electric field position 1030a.

Referring to FIG. 11, FIG. 11 is an application scenario diagram according to an embodiment of the disclosure.

In each scenario of FIG. 11, it is assumed that the driving device 50 drives the third coil 13 with the third pulse current signal S3 having the third signal peak while driving the first coil 11 and the second coil 12. Different from the embodiment of FIG. 10, in FIG. 11, it is assumed that the third coil 13 is driven to provide a third magnetic field of the third magnetic field direction MD3 corresponding to the-Z direction. In this case, the third magnetic field will correspondingly affect the rotating magnetic field formed by the first and second magnetic fields. In other words, the third electric field induced by the third magnetic field will also affect the superimposed electric field formed by the first and second electric fields.

For example, in scenario (a) of FIG. 11, it is assumed that the specified rotating magnetic field direction is the direction of number 4 in Table 1, the driving device 50 may, for example, drive the first coil 11 and the second coil 12 according to the previous teaching to generate a rotating magnetic field with the specified rotating magnetic field direction RMD (such as the direction of number 4 in Table 1), and this rotating magnetic field may correspondingly form a superimposed electric field with the integrated electric field direction RED.

In addition, when the third coil 13 is also driven, the third magnetic field formed by the third coil 13 may cause the rotating magnetic field formed by the first and second magnetic fields to shift toward a specified magnetic field shift position, and the shifted rotating magnetic field (which may be understood as a superimposed magnetic field formed by superimposing the first, second, and third magnetic fields) may correspondingly induce an electric field shown as an electric field diagram 1110.

In the electric field diagram 1110, the electric field shown may be understood as the superimposed electric field of the first and second electric fields after being affected by the third electric field, and may also be understood as a specific superimposed electric field formed by superimposing the first, second, and third electric fields. From the electric field diagram 1110, it may be seen that the specific superimposed electric field shown is shifted towards a specific electric field position 1110a.

From the scenario (a) of FIG. 10 and the scenario (a) of FIG. 11, it may be seen that the electric field may be shifted towards the opposite direction by adjusting the third magnetic field direction MD3 (for example, +Z or −Z direction) of the third magnetic field.

For another example, in scenario (b) of FIG. 11, it is assumed that the specified rotating magnetic field direction is the direction of number 9 in Table 1, the driving device 50 may drive the first coil 11 and the second coil 12 according to the previous teaching to generate a rotating magnetic field with the specified rotating magnetic field direction RMD (for example, the direction of number 9 in Table 1), and this rotating magnetic field may correspondingly form a superimposed electric field with the integrated electric field direction RED.

In addition, when the third coil 13 is also driven, the third magnetic field formed by the third coil 13 may cause the rotating magnetic field formed by the first and second magnetic fields to shift toward the specified magnetic field shift position, and the shifted rotating magnetic field (which may be understood as a superimposed magnetic field formed by superimposing the first, second, and third magnetic fields) may correspondingly induce an electric field shown as an electric field diagram 1120.

In the electric field diagram 1120, the electric field shown may be understood as the superimposed electric field of the first and second electric fields after being affected by the third electric field, and may also be understood as a specific superimposed electric field formed by superimposing the first, second, and third electric fields. From the electric field diagram 1020, it may be seen that the specific superimposed electric field shown is shifted towards a specific electric field position 1120a.

For another example, in scenario (c) of FIG. 11, it is assumed that the specified rotating magnetic field direction is the direction of number 11 in Table 1, the driving device 50 may drive the first coil 11 and the second coil 12 according to the previous teaching to generate a rotating magnetic field with the specified rotating magnetic field direction RMD (for example, the direction of number 11 in Table 1), and this rotating magnetic field may correspondingly form a superimposed electric field with the integrated electric field direction RED.

In addition, when the third coil 13 is also driven, the third magnetic field formed by the third coil 13 may cause the rotating magnetic field formed by the first and second magnetic fields to shift toward the specified magnetic field shift position, and the shifted rotating magnetic field (which may be understood as a superimposed magnetic field formed by superimposing the first, second, and third magnetic fields) may correspondingly induce an electric field shown as an electric field diagram 1130.

In the electric field diagram 1130, the electric field shown may be understood as the superimposed electric field of the first and second electric fields after being affected by the third electric field, and may also be understood as a specific superimposed electric field formed by superimposing the first, second, and third electric fields. From the electric field diagram 1130, it may be seen that the specific superimposed electric field shown is shifted towards a specific electric field position 1130a.

In addition, in scenario (d) of FIG. 11, it is assumed that the specified rotating magnetic field direction is the direction of number 16 in Table 1, the driving device 50 may drive the first coil 11 and the second coil 12 according to the previous teaching to generate a rotating magnetic field with the specified rotating magnetic field direction RMD (for example, the direction of number 16 in Table 1), and this rotating magnetic field may correspondingly form a superimposed electric field with the integrated electric field direction RED.

In addition, when the third coil 13 is also driven, the third magnetic field formed by the third coil 13 may cause the rotating magnetic field formed by the first and second magnetic fields to shift toward a specified magnetic field shift position, and the shifted rotating magnetic field (which may be understood as a superimposed magnetic field formed by superimposing the first, second, and third magnetic fields) may correspondingly induce an electric field shown as an electric field diagram 1140.

In the electric field diagram 1140, the electric field shown may be understood as the superimposed electric field of the first and second electric fields after being affected by the third electric field, and may also be understood as a specific superimposed electric field formed by superimposing the first, second, and third electric fields. From the electric field diagram 1140, it may be seen that the specific superimposed electric field shown is shifted towards a specific electric field position 1140a.

From the scenario (c) of FIG. 10 and the scenario (d) of FIG. 11, it may be seen that the electric field may be shifted to the opposite direction by adjusting the third magnetic field direction MD3 (for example, +Z or −Z direction) of the third magnetic field.

It may be seen from the above, through the method provided by the disclosure, the operator may adjust a specified superimposed electric field formed by the first coil 11, the second coil 12 and the third coil (for example, shift towards a certain direction) by adjusting the first, second and third pulse current signals without moving a position of the transcranial magnetic stimulation 3D coil device 10. In this way, when the transcranial magnetic stimulation 3D coil device 10 is placed on the patient's head, the electric field with a specific direction may be more flexibly applied to the brain area to be treated, thereby improving the convenience of treatment.

In summary, the driving method provided by the embodiment of the disclosure may adjust a field shape/direction/shift position of the superimposed electric field/magnetic field by adjusting the pulse current signal corresponding to each coil without moving the position of the transcranial magnetic stimulation 3D coil device. In this way, when the transcranial magnetic stimulation 3D coil device is placed on the patient's head, the electric field with a specific direction may be more flexibly applied to the brain area to be treated, thereby improving the convenience of treatment.

It will be apparent to those skilled in the art that various modifications and variations can be made to the disclosed embodiments without departing from the scope or spirit of the disclosure. In view of the foregoing, it is intended that the disclosure covers modifications and variations provided they fall within the scope of the following claims and their equivalents.

What is claimed is:

1. A driving method of a transcranial magnetic stimulation 3D coil device, adapted to a driving device, wherein the transcranial magnetic stimulation 3D coil device comprises a plurality of coils, and central axes of each of the coils are perpendicular to each other, the method comprising:
determining a first signal peak and a second signal peak corresponding to a specified rotating magnetic field direction, wherein the first signal peak and the second signal peak respectively correspond to a first coil and a second coil in the coils;
providing a first pulse current signal having the first signal peak to the first coil, wherein the first pulse current signal stimulates the first coil to provide a first magnetic field;
providing a second pulse current signal having the second signal peak to the second coil, wherein the second pulse current signal stimulates the second coil to provide a second magnetic field, and the first magnetic field and the second magnetic field form a rotating magnetic field having the specified rotating magnetic field direction.

2. The driving method according to claim 1, further comprising:
determining a third signal peak, wherein the third signal peak corresponds to a third coil in the coils;
providing a third pulse current signal having the third signal peak to the third coil, wherein the third pulse current signal stimulates the third coil to provide a third magnetic field, and the third magnetic field shifts the rotating magnetic field towards a specified magnetic field shift position.

3. The driving method according to claim 2, wherein the third pulse current signal is a single-phase pulse current signal or a biphasic pulse current signal.

4. The driving method according to claim 2, wherein a third current change rate of the third pulse current signal is 12.53 A/ms to 125.33 A/ms.

5. The driving method according to claim 1, wherein the first pulse current signal and the second pulse current signal are respectively a single-phase pulse current signal or a biphasic pulse current signal.

6. The driving method according to claim 1, wherein a first current change rate of the first pulse current signal is 13.67 A/ms to 136.67 A/ms.

7. The driving method according to claim 1, wherein a second current change rate of the second pulse current signal is 12.89 A/ms to 128.93 A/ms.

8. The driving method according to claim 1, wherein the specified rotating magnetic field direction is selected from K candidate rotating magnetic field directions, and each of the candidate rotating magnetic field directions has a corresponding first reference signal peak and a second reference signal peak, wherein K is a positive integer, and the first reference signal peak and the second reference signal peak corresponding to a jth candidate rotating magnetic field direction in the K candidate rotating magnetic field directions are represented as I_{1,j}(A) and I_{2,j}(A).

9. The driving method according to claim 8, wherein
$$- 1 \leq \frac{{I}_{1 , j} ( A )}{{I}_{1 , max} ( A )} \leq 1 , - 1 \leq \frac{{I}_{2 , j} ( A )}{{I}_{2 , max} ( A )} \leq 1 ,$$
wherein I_{1,max}(A) is a maximum value in the first reference signal peaks corresponding to the each of the candidate rotating magnetic field directions, and I_{2,max}(A) is a maximum value in the second reference signal peaks corresponding to the each of the candidate rotating magnetic field directions.

10. The driving method according to claim 8, wherein
$$- 1.414 \leq \frac{{I}_{2 , j} ( A )}{{I}_{2 , max} ( A )} + \frac{{I}_{1 , j} ( A )}{{I}_{1 , max} ( A )} \leq 1.4 \; 14 ,$$
wherein I_{1,max}(A) is a maximum value in the first reference signal peaks corresponding to the each of the candidate rotating magnetic field directions, and I_{2,max}(A) is a maximum value in the second reference signal peaks corresponding to the each of the candidate rotating magnetic field directions.
